SEQUENCE LISTING (1 sequence)

SEQ ID NO: 1 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

(19) United States
(12) Patent Application Publication
(10) Pub. No.: US 2011/0224138 A1
(43) Pub. Date: Sep. 15, 2011

(54) METHODS FOR TREATING PAIN INDUCED BY INJURIES AND DISEASES OF AN ARTICULAR JOINT

(76) Inventors: Julie Krop, Newton, MA (US); Marilyn Pike, Cambridge, MA (US); Dean Falb, Wellesley, MA (US); Bret Shirley, Waltham, MA (US); Denis Schrier, Hopkinton, MA (US); Mary Elizabeth Goad, Andover, MA (US)

(21) Appl. No.: 12/878,716

(22) Filed: Sep. 9, 2010

Related U.S. Application Data
(60) Provisional application No. 61/240,897, filed on Sep. 9, 2009.

(51) Int. Cl. A61K 38/18; A61P 29/00; A61P 19/08

(52) U.S. Cl. 514/8.8

(57) ABSTRACT

Described herein are methods for using bone morphogenetic proteins (BMPs), such as OP-1 (also known as BMP-7), to treat pain caused by osteoarthritis. The methods involve administering to a patient suffering from pain caused by osteoarthritis a BMP, for example, intraarticularly by injection directly into the joint afflicted with osteoarthritis. The methods of the invention provide long-term pain relief to sufferers of osteoarthritis and can improve the functionality of the joint to which the BMP is administered.

### CROSS-REFERENCE TO RELATED APPLICATIONS

This application claims priority to and the benefit of U.S. Provisional Patent Application No. 61/240,897, filed Sep. 9, 2009, the contents of which are incorporated by reference herein.

### TECHNICAL FIELD OF THE INVENTION

The invention is related to methods for treating pain induced by osteoarthritis that involve administering bone morphogenetic proteins to a patient suffering from pain induced by osteoarthritis. The invention also relates to methods for improving the functionality of joints afflicted by osteoarthritis.

### BACKGROUND

Osteoarthritis (OA), the most common type of joint disease, is a degenerative disorder resulting from the breakdown of articular cartilage in synovial joints (e.g., knee, elbow, wrist, hip, and shoulder). Approximately, 20.7 million Americans suffer from some form of OA and this is expected to increase to 59 million by 2020. Approximately 13% of US adults have OA and it is the leading cause of disability in the elderly. One of the many symptoms associated with OA is joint pain. Typically, osteoarthritic pain is treated by administering to a patient pain-relieving medications such as acetaminophen and anti-inflammatory drugs such as NSAIDs including aspirin or ibuprofen. However, these types of medications must be taken frequently, often multiple times per day, as they do not provide long lasting pain relief for patients with persistent pain. Accordingly, approaches to treating osteoarthritic pain are needed that provide longer term relief, i.e., relief lasting for several weeks or months, preferably with only one dose.

### SUMMARY OF THE INVENTION

Applicants' invention is directed to methods for treating osteoarthritis pain in a joint. Applicants invention is also directed to methods for improving the function of an osteoarthritic joint.

In one embodiment, the invention is a method for treating osteoarthritis pain in a joint of a subject in need thereof. The method comprises the step of administering to the joint a dose of a bone morphogenetic protein effective to relieve the osteoarthritis pain. In one embodiment, the pain is relieved until at least 30 days from the time of administration. In another embodiment, the method is effective to improve functionality in the joint until at least 30 days from the time of administration. In yet another embodiment, the dose of bone morphogenetic protein is not effective to induce substantial cartilage growth in the joint.

According to the invention, in one embodiment, the dose of the BMP is administered by an intraarticular injection to the joint. The joint can be, in one embodiment, a knee joint, in another embodiment, a hip joint, or in another embodiment, a shoulder joint.

According to another embodiment of the invention, the dose of the BMP is effective to relieve pain for at least about 60, about 90, about 120, about 150, about 180 days, or about 360 days from the time of administration of the dose.

In one embodiment, the dose of BMP is a first and only dose. In yet another embodiment, the method further includes the step of administering a second dose of the BMP to the joint at least 60, 90, 120, 150, 180, 210, 240, 270, 300, 330, or 360 days after the first dose, wherein said second dose of the BMP is effective to relieve osteoarthritis pain for at least 30, 60, 90, 120, 150, 180, 210, 240, 270, 300, 330, or 360 days from the time of administration of the second dose. According to one embodiment, the second dose is equal to the first dose.

In yet another embodiment, no further dose follows the first dose of BMP, while in another embodiment, no additional BMP dose is administered to the joint for at least about 60, 90, 120, 150, 180, 210, 240, 270, 300, 330, or 360 days.

According to one embodiment of the invention, the BMP administered is selected from the group consisting of BMP-2, BMP-5, BMP-6, BMP-7, GDF-5, GDF-6 and variants thereof. In one embodiment, the BMP is BMP-7.

According to one embodiment of the invention, the amount of BMP administered is from about 0.01 mg to about 1.0 mg, whereas in another embodiment the amount of BMP administered is from about 0.025 mg to about 0.75 mg. In yet a further embodiment, the amount of BMP administered is from about 0.05 mg to about 0.5 mg, while in yet another embodiment, the amount of BMP administered is from about 0.03 mg to about 0.08 mg, and in yet an even further embodiment, the amount of BMP administered is from about 0.03 mg to about 0.3 mg. In yet another embodiment, the amount of BMP administered is from about 0.08 mg to about 0.4 mg. In a preferred embodiment, the amount of BMP administered is about 0.1 mg.

Another embodiment of the invention is a method for treating pain associated with osteoarthritis in a joint of a subject in need thereof. The method includes the step of intraarticularly administering to the joint a dose of a BMP effective to relieve said pain for at least about 30 days, wherein no additional BMP dose is administered to the joint for at least about 30 days from the date of administration. In another embodiment, the method is further effective to improve functionality in the joint for at least 30 days. In another embodiment, the dose of bone morphogenetic protein is not effective to induce substantial cartilage growth in the joint. In a further embodiment of the method, the amount of BMP administered is between about 0.01 mg and 1.0 mg, while in yet another embodiment, the amount of BMP administered is between about 0.03 mg and 0.3 mg. In an even further embodiment, the amount of BMP administered is about 0.1 mg. In one embodiment, the joint is a knee joint. In one embodiment, the subject is a human.

Yet another embodiment of the invention is a method for reducing pain in an osteoarthritic joint of a patient suffering from osteoarthritis. The method includes the step of administering to the patient a single dose of a BMP. The first dose is effective to relieve the osteoarthritis pain until at least 30 days from the time of administration. According to one embodiment, a further dose, if administered, is not administered until at least 30 days or at least 60 days or at least 90 days, or least 180 days, or at least 360 days from the administration of the first dose. According to one embodiment, the single dose is injected into the osteoarthritic joint intraarticularly. The BMP can be OP-1. The single dose can be about 0.03 mg to about 0.3 mg of BMP or, in another embodiment, the single dose can be about 0.1 mg of BMP. In a further embodiment, the joint is a knee joint.

### BRIEF DESCRIPTION OF THE FIGURES

FIG. 1 is a table showing data collected from all study cohorts (0.03, 0.1, 0.3, and 1.0 mg OP-1 and placebo) indicating changes from baseline values (Day 1) of the KOOS pain subscale over the course of 4 weeks, 8 weeks, 12, weeks and 24 weeks post-treatment as experienced by the patients in the treatment knee.

FIG. 2 is a table showing data collected from all study cohorts (0.03, 0.1, 0.3, and 1.0 mg OP-1 and placebo) indicating the number of patients experiencing 20%, 50% and 70% reduction in WOMAC pain subscale values from baseline values (Day 1) over the course of 4 weeks, 8 weeks, 12 weeks and 24 weeks post-treatment as experienced by the patients in the treatment knee.

FIG. 3 is a line graph showing the mean % change from baseline of WOMAC pain values as experienced by patients administered 0.1 mg OP-1 on Day 1 versus the placebo group as measured at 4 weeks, 8 weeks, 12 weeks, and 24 weeks.

FIG. 4 is a table showing data collected from all study cohorts (0.03, 0.1, 0.3, and 1.0 mg OP-1 and placebo) indicating changes from baseline values (Day 1) of the KOOS function in daily living subscale over the course of 4 weeks, 8 weeks, 12 weeks and 24 weeks post-treatment as experienced by the patients in the treatment knee.

FIG. 5 is a table showing data collected from all study cohorts (0.03, 0.1, 0.3, and 1.0 mg OP-1 and placebo) indicating the number of patients experiencing 20%, 50% and 70% improvement in WOMAC function subscale values from baseline values (Day 1) over the course of 4 weeks, 8 weeks, 12 weeks and 24 weeks post-treatment as experienced by the patients in the treatment knee.

FIG. 6 is a table showing data collected from all study cohorts (0.03, 0.1, 0.3, and 1.0 mg OP-1 and placebo) indicating changes from baseline values (Day 1) for the Patient Global Assessment of Disease Status Visual Analog Scale (VAS) as determined for the treatment knee.

FIG. 7 is a table showing data collected from all study cohorts (0.03, 0.1, 0.3, and 1.0 mg OP-1 and placebo) indicating changes from baseline values (Day 1) for the Physician Global Assessment of Disease Status Visual Analog Scale (VAS) as determined for the treatment knee.

FIG. 8 is a standard curve showing alkaline phosphatase activity as a function of the log of BMP-7 concentration (ng/mL).

FIG. 9 shows the amino acid sequence of mature human BMP-7.

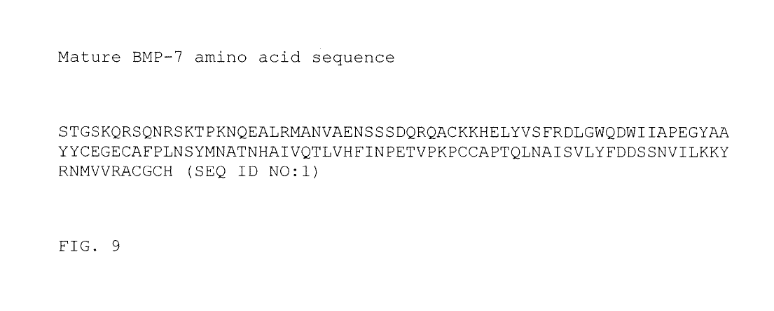

### DETAILED DESCRIPTION OF THE INVENTION

Applicants were the first to discover that administration of bone morphogenetic proteins (BMPs) to joints of patients suffering from osteoarthritis (OA) can relieve the pain caused by this degenerative disease when administered in an appropriate amount according to a particular dosing schedule. While BMPs have been shown in non-human animal models to have reparative effects on cartilage when administered long-term to joints afflicted with osteoarthritis, such reparative effects are achieved only when BMPs are administered continuously (e.g., via an implanted pump) or periodically at specific frequent intervals over a course of days, weeks, or months.

According to Applicants' invention, patients experience a reduction in osteoarthritis induced pain when BMPs are administered at doses significantly lower than cumulative doses that would be expected to result in a reparative effect on cartilage in humans and when administered at significantly lesser frequencies than would be expected to result in a reparative effect on cartilage in humans. Moreover, pain reduction is observed in the short term and persists for the long term.

Further, according to one aspect of Applicants' invention, patients experience a reduction in pain with a concomitant improvement in function. Generally speaking, pain relief does not always correlate with an improvement in function, but in this case, Applicants' invention was effective in providing an improvement in function as demonstrated by the data discussed herein, which was also unexpected.

As shown by the data presented herein, Applicants discovered that a single dose of the exemplary bone morphogenetic protein OP-1 (also known as BMP-7) provided the overall best reduction in pain and for the longest time period, e.g., at least 6 months. This result was completely unexpected as it was initially predicted that sustained exposure over weeks or months through multiple doses, either daily or weekly, or a sustained release formulation would be required to achieve the levels and duration of pain relief actually seen by Applicants when a single dose of OP-1 was administered. The best effect was seen with a single dose of 0.1 mg OP-1 as prepared according to Example 2 provided herein. Further, this effect was not seen with the higher dose of 1.0 mg or doses lower than 0.1 mg (prepared according to Example 2) indicating that the amount of OP-1 required to achieve this effect is highly dose specific and that increasing the dose does not enhance the pain relieving affect experienced.

### Bone Morphogenic Proteins

According to Applicants' invention, bone morphogenetic proteins (BMPs) are the preferred exemplary protein administered to patients suffering from osteoarthritis induced pain in order to treat and reduce the pain experienced by the patients. BMPs belong to the TGF-β superfamily. The TGF-β superfamily proteins are cytokines characterized by six-conserved cysteine residues. The human genome contains about 42 open reading frames encoding TGF-β superfamily proteins. The TGF-β superfamily proteins can at least be divided into the BMP subfamily and the TGF-β subfamily based on sequence similarity and the specific signaling pathways that they activate. The BMP subfamily includes, but is not limited to, BMP-2, BMP-3 (osteogenin), BMP-3b (GDF-10), BMP-4 (BMP-2b), BMP-5, BMP-6, BMP-7 (osteogenic protein-1 or OP-1), BMP-8 (OP-2), BMP-8B (OP-3), BMP-9 (GDF-2), BMP-10, BMP-11 (GDF-11), BMP-12 (GDF-7), BMP-13 (GDF-6, CDMP-2), BMP-15 (GDF-9), BMP-16, GDF-1, GDF-3, GDF-5 (CDMP-1, MP-52), and GDF-8 (myostatin). For purposes of the present invention, preferred superfamily proteins for administration according to the invention disclosed herein include BMP-2, -4, -5, -6 and -7 and GDF-5, -6, and -7, as well as MP-52. Particularly preferred proteins include BMP-2, BMP-5, BMP-6, BMP-7 and GDF-5, -6, and -7. A most preferred exemplary BMP for administration to a patient according to the invention is human BMP-7 or OP-1. Furthermore, there is allelic variation in BMP sequences among different members of the human population, and there is species variation among BMPs discovered and characterized to date. As used herein, “BMP subfamily,” “BMPs,” “BMP ligands” and grammatical equivalents thereof refer to the BMP subfamily members, unless specifically indicated otherwise. It is contemplated that any of the members of the BMP subfamily disclosed herein can be administered to a patient suffering from pain induced by osteoarthritis in order to reduce the pain caused by osteoarthritis according to the methods of the invention disclosed herein. In one embodiment, the BMP is OP-1 (BMP-7). In another embodiment, the BMP is BMP-2. In another embodiment, the BMP is BMP-6. In another embodiment, the BMP is BMP-5.

Publications disclosing these sequences, as well as their chemical and physical properties, include: BMP-7 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)), BMP-2, BMP-4, (WO88/00205; Wozney et al. Science, 242, pp. 1528-1534 (1988)), BMP-5 and BMP-6, (Celeste et al., PNAS, 87, 9843-9847 (1990)), Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/U595/07084); BMP-10 (WO94/26893 (PCT/US94/05290); BMP-11 (WO94/26892 (PCT/US94/05288); BMP-12 (WO95/16035 (PCT/US94/14030); BMP-13 (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-11 (WO96/01845 (PCT/US95/08543); BMP-15 (WO96/36710 (PCT/US96/06540); MP-121 (WO96/01316 (PCT/EP95/02552); GDF-5 (CDMP-1, MP52) (WO94/15949 (PCT/US94/00657) and WO96/14335 (PCT/US94/12814) and WO93/16099 (PCT/EP93/00350)); GDF-6 (CDMP-2, BMP13) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)) The above publications are incorporated herein by reference.

As used herein, “TGF-β superfamily member” or “TGF-β superfamily protein,” means a protein known to those of ordinary skill in the art as a member of the Transforming Growth Factor-0 (TGF-β) superfamily. Structurally, such proteins are homo or heterodimers expressed as large precursor polypeptide chains containing a hydrophobic signal sequence, an N-terminal pro region of several hundred amino acids, and a mature domain comprising a variable N-terminal region and a highly conserved C-terminal region containing approximately 100 amino acids with a characteristic cysteine motif having a conserved six or seven cysteine skeleton. These structurally-related proteins have been identified as being involved in a variety of developmental events.

The term “morphogenic protein” refers to a protein belonging to the TGF-β superfamily of proteins which has true morphogenic activity. For instance, such a protein is capable of inducing progenitor cells to proliferate and/or to initiate a cascade of events in a differentiation pathway that leads to the formation of cartilage, bone, tendon, ligament, neural or other types of differentiated tissue, depending on local environmental cues. Thus, morphogenic proteins useful according to the invention can behave differently in different surroundings. In certain embodiments, a morphogenic protein of this invention can be a homodimer species or a heterodimer species.

The term “osteogenic protein (OP)” refers to a morphogenic protein that is also capable of inducing a progenitor cell to form cartilage and/or bone. The bone can be intramembranous bone or endochondral bone. Most osteogenic proteins are members of the BMP subfamily and are thus also BMPs. However, the converse can not be true. According to this invention, a BMP identified by DNA sequence homology or amino acid sequence identity must also have demonstrable osteogenic or chondrogenic activity in a functional bioassay to be an osteogenic protein. Appropriate bioassays are well known in the art; a particularly useful bioassay is the heterotopic bone formation assay (see, U.S. Pat. No. 5,011,691; U.S. Pat. No. 5,266,683, for example).

Structurally, BMPs are dimeric cysteine knot proteins. Each BMP monomer comprises multiple intramolecular disulfide bonds. An additional intermolecular disulfide bond mediates dimerization in most BMPs. BMPs can form homodimers. Some BMPs can form heterodimers. BMPs are expressed as pro-proteins comprising a long pro-domain, one or more cleavage sites, and a mature domain. The pro-domain is believed to aid in the correct folding and processing of BMPs. Furthermore, in some but not all BMPs, the pro-domain can noncovalently bind the mature domain and can act as an inhibitor (e.g., Thies et al. (2001) Growth Factors 18:251-259).

BMPs are naturally expressed as pro-proteins comprising a long pro-domain, one or more cleavage sites, and a mature domain. This pro-protein is then processed by the cellular machinery to yield a dimeric mature BMP molecule. The pro-domain is believed to aid in the correct folding and processing of BMPs. Furthermore, in some but not all BMPs, the pro-domain can noncovalently bind the mature domain and can act as a chaperone, as well as an inhibitor (e.g., Thies et. al. (2001) Growth Factors, 18:251-259).

BMP signal transduction is initiated when a BMP dimer binds two type I and two type II serine/threonine kinase receptors. Type I receptors include, but are not limited to, ALK-1, ALK-2 (also called ActRla or ActRI), ALK-3 (also called BMPRIa), and ALK-6 (also called BMPRIb). Type II receptors include, but are not limited to, ActRIIa (also called ActRII), ActRIIb, and BMPRII. Human genome contains 12 members of the receptor serine/threonine kinase family, including 7 type I and 5 type II receptors, all of which are involved in TGF-β signaling (Manning et al., 2002, Science, 298:1912-1934) the disclosures of which are hereby incorporated by reference). Following BMP binding, the type II receptors phosphorylate the type I receptors, the type I receptors phosphorylate members of the Smad family of transcription factors, and the Smads translocate to the nucleus and activate the expression of a number of genes.

BMPs also interact with inhibitors, soluble receptors, and decoy receptors, including, but not limited to, BAMBI (BMP and activin membrane bound inhibitor), BMPER (BMP-binding endothelial cell precursor-derived regulator), Cerberus, cordin, cordin-like, Dan, Dante, follistatin, follistatin-related protein (FSRP), ectodin, gremlin, noggin, protein related to Dan and cerberus (PRDC), sclerostin, sclerostin-like, and uterine sensitization-associated gene-1 (USAG-1). Furthermore, BMPs can interact with co-receptors, for example BMP-2 and BMP-4 bind the co-receptor DRAGON (Samad et. al. (2005) J. Biol. Chem., 280:14122-14129), and extracellular matrix components such as heparin sulfate and heparin (Irie et al. (2003) Biochem. Biophys. Res. Commun. 308: 858-865).

As contemplated herein, the term “BMP” refers to a protein belonging to the BMP subfamily of the TGF-β superfamily of proteins defined on the basis of DNA homology and amino acid sequence identity. According to this invention, a protein belongs to the BMP subfamily when it has at least 50% amino acid sequence identity with a known BMP subfamily member within the conserved C-terminal cysteine-rich domain that characterizes the BMP subfamily. Members of the BMP subfamily can have less than 50% DNA or amino acid sequence identity overall. As used herein, the term “BMP” further refers to proteins which are amino acid sequence variants, domain-swapped variants, and truncations and active fragments of naturally occurring bone morphogenetic proteins, as well as heterodimeric proteins formed from two different monomeric BMP peptides, such as BMP-2/7; BMP-4/7: BMP-2/6; BMP-2/5; BMP-4/7; BMP-4/5; and BMP-4/6 heterodimers. Suitable BMP variants and heterodimers include those set forth in US 2006/0235204; WO 07/087,053; WO 05/097825; WO 00/020607; WO 00/020591; WO 00/020449; WO 05/113585; WO 95/016034 and WO93/009229.

According to one embodiment, a BMP used according to the methods of the invention can maintain at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with the corresponding wild-type BMP protein sequence.

According to one embodiment, a BMP used according to the methods of the invention can maintain at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with the conserved cysteine domain of the C-terminal region of the corresponding wild-type BMP protein sequence.

By “corresponding wild-type protein” it is meant the wild-type version of the modified BMP. For example, if the modified BMP is a modified BMP-7, the corresponding wild-type BMP is wild-type BMP-7.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=# of identical positions/total # of positionsX100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al. (1990) J. Mol. Biol. 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used.

### Administration and Dosing

According to the invention, Applicants have discovered that osteoarthritic patients can experience a significant reduction in pain when they are treated with a single dose of bone morphogenetic protein (BMP), for example, BMP-7, rather than multiple doses predicted to be necessary to demonstrate a reparative or regenerative effect on cartilage in humans. The single dose administers a much lower amount of BMP to the patient in comparison to the cumulative total of BMP administered to a patient in a multiple dosing regimen.

While it has been shown in non-human animal models that bone morphogenetic proteins need to be administered consistently over days or weeks to induce cartilage growth, Applicants have discovered that pain reduction occurs with a single dose of BMP which is a smaller cumulative amount of BMP than what was thought to be necessary to induce cartilage repair in humans. Further, Applicants have discovered that pain reduction occurs with only a single dose of BMP and that the reduction in pain from the single dose lasts for several months.

The invention provides methods to treat osteoarthritis pain, that is, pain caused by osteoarthritis. For example, according to one embodiment, the invention includes a method for treating osteoarthritis pain in a joint of a subject in need thereof comprising the step of administering to the joint a dose of bone morphogenetic protein effective to relieve said osteoarthritis pain.

According to one embodiment, the administration of BMP is not effective to induce substantial cartilage growth, repair, or restoration.

Treating osteoarthritis pain according to one embodiment of the invention means eliminating or substantially eliminating pain. According to another embodiment, treating osteoarthritis pain means reducing the level of pain experienced by a patient. In another embodiment, reduction of pain is measured by reference to a baseline of pain experienced by the patient prior to the patient being treated with a bone morphogenetic protein according to the invention. For example, in one embodiment, the patient experiences a reduction of pain based on the commonly known KOOS (Knee and Osteoarthritis Outcome Score) pain subscale score which quantifies a patient's experience of pain based on a known range of factors (see, e.g., Roos et al., J. Orthop. Sports. Phys. Ther., (1998) 28:22-96). For example, in one embodiment, the patient experiences at least a 5, 10, 15, 20, 25, 30, 35, 40, 45, or 50 point change from baseline based on the KOOS pain subscale. In another embodiment, the patient experiences a reduction of pain measured by the commonly known WOMAC (Western Ontario and MacMaster Universities Osteoarthritis Index) pain subscale (see, e.g., Bellamy et al., Ann. Rheum. Dis., (2005), 64:881-885). For example, in one embodiment, the patient experiences at least a 20%, 30%, 40%, 50%, 60%, 70%, 80%, 80% or 100% reduction in pain from baseline based on the WOMAC pain subscale.

According to one embodiment, the invention provides methods to improve the functionality of an osteoarthritic joint. Accordingly, the invention provides a method for improving the functionality of an osteoarthritic joint by administering a BMP to the joint, or in another embodiment, the invention provides a methods for reducing the pain in a joint and improving the functionality of the joint by administering a BMP to the joint.

In one embodiment, improvement in functionality is measured by reference to a baseline of functionality experienced by the patient prior to the patient being treated with a BMP according to the invention. For example, in one embodiment, the patient experiences an improvement in functionality based on the commonly known KOOS (Knee and Osteoarthritis Outcome Score) function in daily living subscale which quantifies a joint's functionality based on a known range of factors (see, e.g., Roos et al., J. Orthop. Sports. Phys. Ther., (1998) 28:22-96). For example, in one embodiment, the patient experiences at least a 5, 10, 15, 20, 25, 30, 35, 40, 45, or 50 point change from baseline based on the KOOS function in daily living subscale. In another embodiment, the patient experiences an improvement in function measured by the commonly known WOMAC (Western Ontario and MacMaster Universities Osteoarthritis Index) function subscale (see, e.g., Bellamy et al., Ann. Rheum. Dis., (2005), 64:881-885). For example, in one embodiment, the patient experiences at least a 20%, 30%, 40%, 50%, 60%, 70%, 80%, 80% or 100% improvement in function from baseline based on the WOMAC pain subscale.

In addition to treating pain caused by osteoarthritis, according to another embodiment of the invention, a BMP can be administered to a patient to treat pain caused by any disease or damage to articular cartilage or an osteochondral defect or disease. Accordingly, the methods of this invention are not limited to merely treating pain and restoring loss of functionality caused by osteoarthritis.

According to one embodiment of the invention, the BMP is administered directly to the site where the patient experiences pain. For example, the BMP can be administered directly to a knee joint, hip joint, finger or thumb joint, toe joint, ankle joint, wrist joint, shoulder joint, elbow joint or joints of the spine such as facet joints. In a preferred embodiment, the BMP is administered to a knee joint. In another embodiment, the BMP is administered to a hip joint.

According to one embodiment of the invention, the BMP is administered to the patient intraarticularly, i.e., directly into the joint. For example, the BMP is administered via injection through a syringe and needle. According to another embodiment, the BMP is administered topically at the site of the joint. In another embodiment, the BMP is administered subcutaneously or intravenously.

According to one embodiment of the invention, the BMP is administered to a patient only once in order to reduce the pain caused by osteoarthritis. For example, in one embodiment, the BMP is administered on Day 1 and no further administration of BMP to the joint occurs thereafter. According to the invention, patients experience pain relief or a reduction in pain with this single dose that lasts for at least 30 days, at least 60 days, at least 90 days, at least 120 days, at least 150 days, at least 180 days, at least 360 days, or at least 720 days with out receiving a further dose. In yet another embodiment, the BMP is administered to a patient and no additional dose of BMP is administered to the patient for at least about 30 days, about 60 days, about 90 days, about 120 days, about 150 days, about 180 days, about 210 days, about 240 days, about 270 days, about 300 days, about 330 days, or about 360 days after the first administration. In a further embodiment, an additional dose of BMP is effective to treat osteoarthritis pain in a patient for at least 30 days, at least 60 days, at least 90 days, at least 120 days, at least 150 days, at least 180 days, or at least 360 days. For example, in one embodiment, a first dose of BMP is administered to a patient's joint and no further administration occurs for 180 days and the dose is effective to treat osteoarthritis pain in the patient's joint for 180 days.

According to another embodiment of the invention, the dose of BMP is between about 0.01 mg and about 1.0 mg. In a further embodiment, the dose of BMP is between about 0.025 mg and about 0.75 mg. In yet another embodiment, the dose of BMP is between about 0.05 mg and about 0.5 mg. In yet another embodiment, the dose of BMP is between about 0.03 mg to about 0.08 mg. In yet another embodiment, the dose of BMP is between about 0.03 mg and about 0.3 mg. In yet another embodiment, the dose of BMP is between about 0.08 mg and about 0.4 mg. In yet another embodiment, the dose is between about 0.1 mg and about 0.3 mg. In another embodiment, the dose is about 0.1 mg BMP. In another embodiment, the dose is about 0.3 mg. The aforementioned doses can be administered as a first and only dose according to the invention, or they can be administered as a second or supplemental dose according to the methods of the invention described herein. In another embodiment, the aforementioned dose ranges are appropriate for administration to a knee. In another embodiment, dosages appropriate for administration to joints other than the knee can be determined based on comparing the surface area of the joint, the volume of synovial fluid in the joint, and the weight of the patient in comparison to that of the knee and adjusting the dosage proportionally based on the larger or smaller size of the joint.

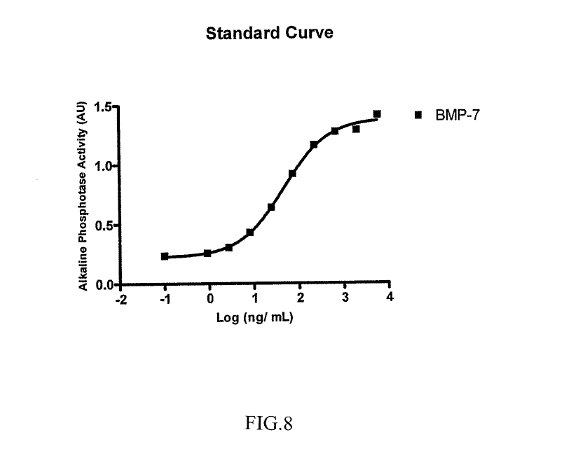

According to one embodiment of the invention, the dosages described herein are determined based on the activity level of an exemplary BMP, recombinant OP-1 as provided, for example, in Example 2. According to another embodiment of the invention, the dosages described herein are adjusted based on activity level of a BMP protein and may be increased proportionally if the activity level is less than that of the BMP protein described in Example 2 or may decreased proportionally if the activity level is more than that of the BMP protein described in Example 2. For example, the alkaline phosphatase activity can be determined for a given BMP in accordance with the protocol outlined in Example 1. To determine whether more or less of the BMP should be used in a dose than the doses described herein, the alkaline phosphatase activity for a certain concentration of BMP can be determined and compared to the alkaline phosphatase activities provided in the standard curve in FIG. 8 for OP-1 as prepared according to Example 2. The dosage can be adjusted to reduce or increase the amount of BMP so that the total activity of the dosage is equivalent to that provided by a dose of OP-1 prepared in Example 2.

According to another embodiment of the invention, the BMP is administered in a liquid, gel, or paste form. For example, in a preferred embodiment, the BMP is administered in liquid form, for example, an aqueous solution. In one embodiment, the BMP is reconstituted from a lyophilized form with an aqueous buffer prior to administration. For example, the buffer may be a lactate buffer. Other suitable buffers include acetate, citrate, trifluoroacetate, glycine, and phosphate buffers. The solution can also include a lyoprotectant or stabilizing agent. Suitable lyoprotectants include sugars and sugar alcohols, such as sucrose, lactose, trehalose, and mannitol. In one embodiment, the solution can include other excipients known in the art for stabilizing proteins formulations. In one embodiment, the lyophilized protein composition also includes buffer salts and, optionally, a lyoprotectant, and can be reconstituted with sterile water, such as water for injection. In one embodiment, the total dose of BMP is delivered to the patient as a liquid, gel or paste with a total volume of 1.0 mL. In another embodiment, the total dose of BMP is delivered to the patient as a liquid, gel or paste with a total volume of 0.5 mL, 1.5 mL, or 2.0 mL. In one embodiment, the BMP formulation is not a sustained release or sustained activity formulation.

### Example 1

### Determining the Alkaline Phosphatase Activity of OP-1

In order to determine the alkaline phosphatase activity of OP-1 (BMP-7), Ros 17/2.8 Cells were seeded in a 96 well plate at 3.7×10⁵ cells per well and incubated at 37° C., 5% CO₂ for 24 hours. Bulk OP-1 reference standard (Stryker Biotech, Hopkinton, Mass.) and samples were diluted to 30 μg/ml and serial diluted 6-fold with F-12 BSA. 50 μl (30 μg/ml) of reference standard and test samples were added to the cells which contained 200 μl of medium. The final protein concentration on the cells is 6 μg/ml diluted 6-fold as shown in the plate map in Table 1 below. The reference standard and the test samples were incubated on the cells for 24 hours at 37° C., 5% CO₂. After incubation 150 μA of waste medium was removed from the cells and 100 μA of 2% Triton X-100 was added to lyse the cells. The lysed cells were incubated at 37° C., 5% CO₂ for 1 hour.

The lysed plates were spun at 2600 RPM for 10 mins. 20 μA of the lysate was removed and transferred to a clear plate. 100 μA of 1:5 pNitrophenyl phosphate (pNPP) was added for 10 min and incubated at 37° C., 5% CO₂. After incubation time the alkaline phosphatase activity reaction was stopped by using 75 μA of 0.5 N NaOH. The plates were read using a SPECTRAmax Microplate Reader. A standard curve showing alkaline phosphatase activity as a function of OP-1 (BMP-7) concentration is shown in FIG. 8.

TABLE 1
Plate Map

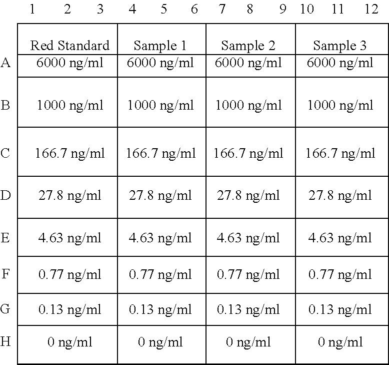

### Example 2

### Preparing an OP-1 Formulation for Administration to OA Patients

Recombinant human OP-1 (also known as BMP-7 or by the International Non-proprietary Name eptotermin alfa) was supplied as 1.0 mg lyophilized cake in 6 mL vials (Stryker Biotech LLC, Hopkinton, Mass.). Each cake was reconstituted with 1.0 mL sterile water resulting in a solution containing 1.0 mg/mL OP-1 in 5% lactose (weight/volume). Vials of OP-1 were diluted as needed with 5% lactose solution in order to arrive at 0.03 mg/mL, 0.1 mg/mL, and 0.3 mg/mL respectively.

### Example 3

### Administration of OP-1 Formulation to a Patient

Recombinant human OP-1 or a placebo was administered to 33 subjects in 4 cohorts. Six subjects in each cohort were randomly assigned to receive 0.3 mg (7 subjects), 0.1 mg, 0.3 mg, or 1.0 mg of OP-1. Two subjects in each cohort were randomly assigned to receive a placebo of 1 mL 5% lactose. On day 1, a total volume of 1.0 mL containing the specified dose or placebo for each study cohort was injected intraarticularly into the knee using a 3 mL syringe under ultrasound or fluoroscopy guidance to ensure injection into the knee joint. Subjects underwent a 168 day (24 week) follow-up period. No further administration of OP-1 or placebo occurred after the initial injection on Day 1.

### Example 4

### Results of OP-1 Administration on the KOOS Pain Subscale

Patients were assessed 4 weeks, 8 weeks, 12 weeks, and 24 weeks from the first administration to determine changes in their “KOOS” score (Knee and Osteoarthritic Outcome Score). The KOOS pain subscale ranged from 100 which means “no problems” to 0 which means “extreme problems.” Positive changes from baseline indicated improvement and negative changes indicated worsening.

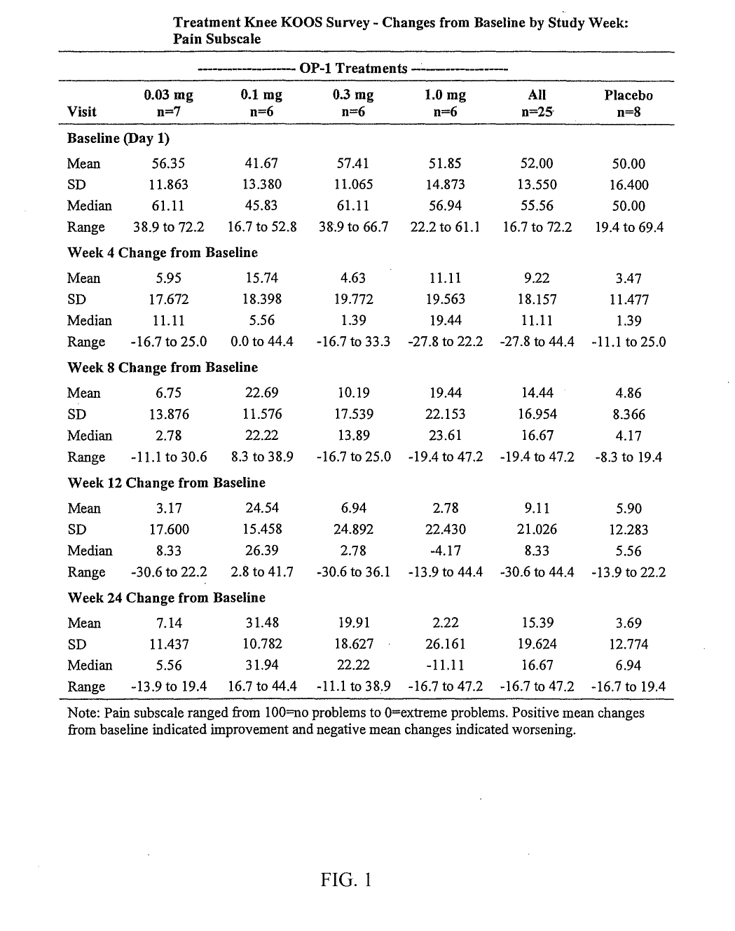

FIG. 1 provides a summary of the results of the pain subscale of the KOOS survey and provides comparisons of the KOOS pain subscale for each of the treatment groups to the placebo group. At Week 4, the mean change from baseline for the 0.03, 0.1, 0.3 and 1.0 mg OP-1 groups was higher than the mean change from baseline for the placebo group. At Week 8, the mean change from baseline for the 0.03, 0.1, 0.3, and 1.0 mg OP-1 groups was higher than the mean change from baseline for the placebo group. At week 12, the mean change from baseline for the 0.1 and 0.3 mg OP-1 groups was higher than the mean change from baseline for the placebo group. The mean change from baseline for the 0.03 mg and 1.0 mg OP-1 groups was lower than the mean change for the placebo group. At week 24, the mean change from baseline for the 0.03, 0.1, and 0.3 mg groups was higher than the mean change from baseline for the placebo group. The mean change from baseline for the 1.0 mg OP-1 groups was lower than the mean change for the placebo group.

As shown by the data in FIG. 1, the patients who were administered 0.1 mg of OP-1 saw the greatest positive mean changes from baseline over time, the improvement continuing all the way through week 24. This trend of improvement in pain cessation over the course of 24 weeks was not seen with 0.03 mg, 0.3 mg, or 1.0 mg doses. At weeks 4 and 8, the mean change from baseline for all groups was higher than the mean change from baseline for the placebo group. In summary, the effects of the OP-1 treatment on KOOS pain subscale indicated that the 0.03 mg group was similar to the placebo group; that the 1.0 mg OP-1 group showed improvement at weeks 4 and 8, but the baseline waned at weeks 12 and 24, with 50% of subjects being worse off than the baseline; and the 0.1 mg OP-1 group showed the most improvement, with improvements being consistent through week 24.

### Example 5

### Results of OP-1 Administration on the KOOS Function in Daily Living Subscale

Patients were assessed 4 weeks, 8 weeks, 12 weeks, and 24 weeks from the first administration to determine changes in their “KOOS” score (Knee and Osteoarthritic Outcome Score). The KOOS pain subscale ranged from 100 which means “no problems” to 0 which means “extreme problems.” Positive changes from baseline indicated improvement and negative changes indicated worsening.

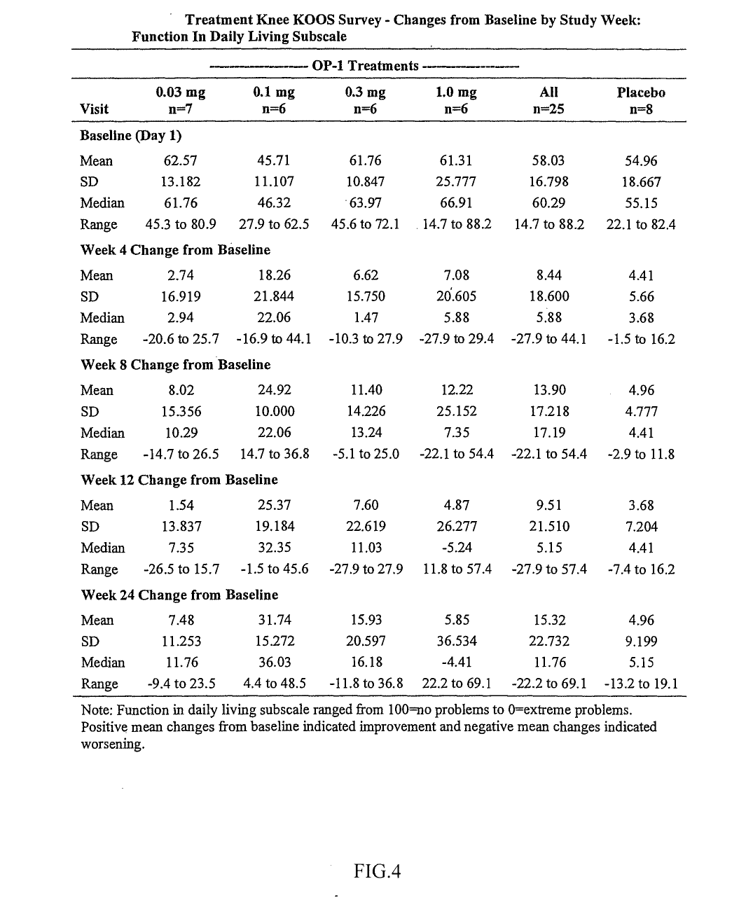

FIG. 4 provides a summary of the results of the function in daily living subscale of the KOOS survey and provides comparisons of the KOOS function in daily living for each of the treatment groups to the placebo group. At Week 4, the mean change from baseline for the 0.1, 0.3 and 1.0 mg OP-1 groups was higher than the mean change from baseline for the placebo group. The mean change from baseline for the 0.03 mg OP-1 group was lower than the mean change for the placebo group. At Week 8, the mean change from baseline for the 0.03, 0.1, 0.3, and 1.0 mg OP-1 groups was higher than the mean change from baseline for the placebo group. At Week 12, the mean change from baseline for the 0.1, 0.3, and 1.0 mg OP-1 groups was higher than the mean change from baseline for the placebo group. The mean change from baseline for the 0.03 OP-1 group was lower than the mean change for the placebo group. At week 24, the mean change from baseline for the 0.03, 0.1, 0.3, and 1.0 mg groups was higher than the mean change from baseline for the placebo group.

In summary, the effects of the OP-1 treatment on KOOS function in daily living subscale indicated that the 0.1 mg group showed the most improvement in function, that the 0.3 mg group showed good improvement, and that while the 1.0 mg group showed improvement early on, the improvement waned at weeks 12 and 24. The greatest improvements in functionality experienced can be correlated to greatest reduction in pain as shown by the data for the 0.1 mg group on both KOOS pain subscale and function in daily living subscale. As previously discussed, the improvements in pain and functionality were not expected.

### Example 6

### Results of OP-1 Administration on the WOMAC Pain Subscale

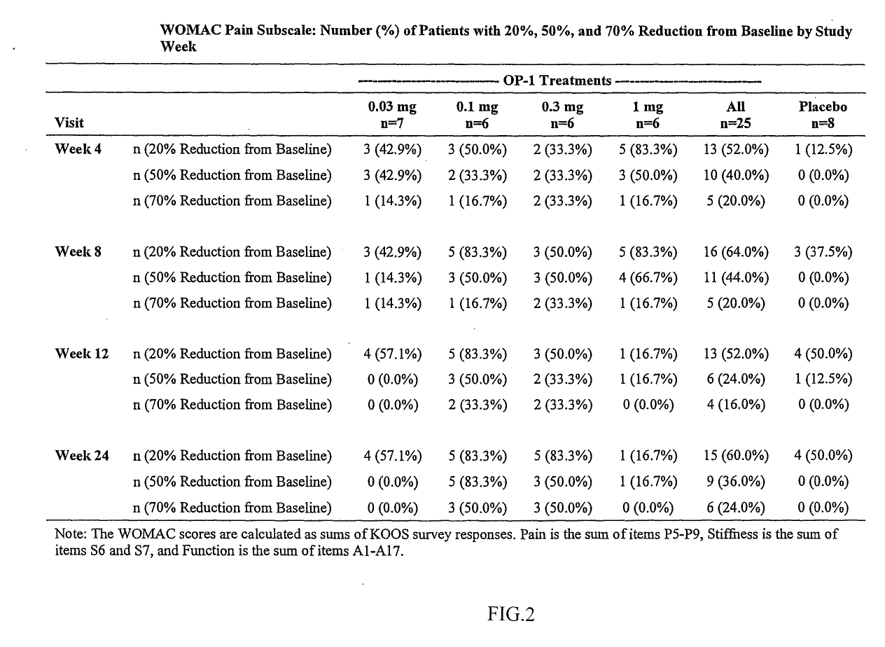

Patients were assessed 4 weeks, 8 weeks, 12 weeks, and 24 weeks from the first administration to determine changes in their “WOMAC” score (Western Ontario and McMaster Universities Osteoarthritis Index). The WOMAC pain subscale values are presented as absolute values and percentage changes from baseline where an increase in the score indicates a worsening of a clinical parameter and a decrease indicates an improvement in a clinical parameter. The proportion of subjects with a 20%, 50%, and 70% reduction in the WOMAC pain subscale at 4, 8, 12, and 24 weeks is summarized in FIG. 2.

At Weeks 4 and 8, the 0.03, 0.1, 0.3, and 1.0 mg OP-1 groups all had a greater proportion of subjects that achieved a 20% reduction from baseline than the placebo group. At Week 12, the 0.03 and 0.1 mg OP-1 groups had a greater proportion of subjects that achieved a 20% reduction than the placebo group. The proportion for the 1.0 mg OP-1 group was lower than the placebo group. At Week 24, the 0.03, 01, and 0.3 mg OP-1 groups had a greater proportion of subjects that achieved a 20% reduction than the placebo group. The proportion for the 1.0 mg OP-1 group was lower than the placebo group.

At Weeks 4 and 8, the 0.03, 0.1, 0.3, and 1.0 mg OP-1 groups all had a greater proportion of subjects that achieved a 50% reduction from baseline than the placebo group. At Week 12, the 0.1, 0.3, and 1.0 mg OP-1 groups had a greater proportion of subjects that achieved a 50% reduction than the placebo group. The proportion for the 0.03 mg OP-1 group was lower than the placebo group. At Week 24, the 0.1, 0.3, and 1.0 mg OP-1 groups had a greater proportion of subjects that achieved a 50% reduction than the placebo group. The proportion for the 0.03 mg OP-1 group was the same as the placebo group.

At Weeks 4 and 8, the 0.03, 0.1, 0.3, and 1.0 mg OP-1 groups all had a greater proportion of subjects that achieved a 70% reduction from baseline than the placebo group. At Week 12, the 0.1 mg and 0.3 mg OP-1 groups had a greater proportion of subjects that achieved a 70% reduction than the placebo group. The proportion for the 0.03 and 1.0 mg OP-1 groups was the same as the placebo group. At Week 24, the 0.1 and 0.3 mg groups had a greater proportion of subjects that achieved a 70% reduction than the placebo group. The proportion for the 0.03 and 1.0 mg OP-1 groups was the same as the placebo group.

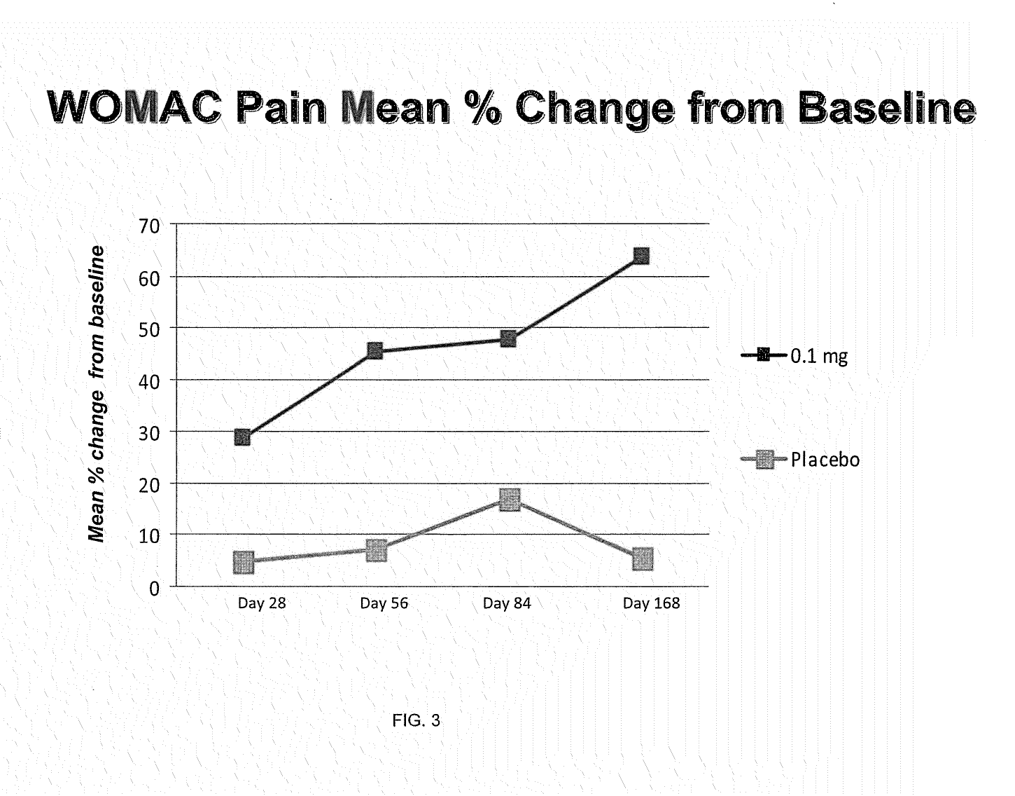

In summary, as shown by the data in FIG. 2, most subjects, including the placebo subjects, experienced a 20% improvement in knee pain by Week 12. However, the groups receiving OP-1 had a higher proportion than placebo experiencing a 50% and 70% reduction in knee pain. More importantly, those subjects receiving 0.1 mg or 0.3 mg maintained the 50% and 70% reduction in pain through Week 24 while those receiving other administrations did not. In fact, those receiving 0.03 or 1 mg or OP-1 did not see maintenance of 50% and 70% reductions in pain past about 8 weeks. The mean % reduction in pain from baseline for the 0.1 mg group versus placebo is also summarized in FIG. 3 which shows that for those administered 0.1 mg, the % reduction continued over time and improvements were maintained through to Week 24.

### Example 7

### Results of OP-1 Administration on the WOMAC Pain Function Subscale

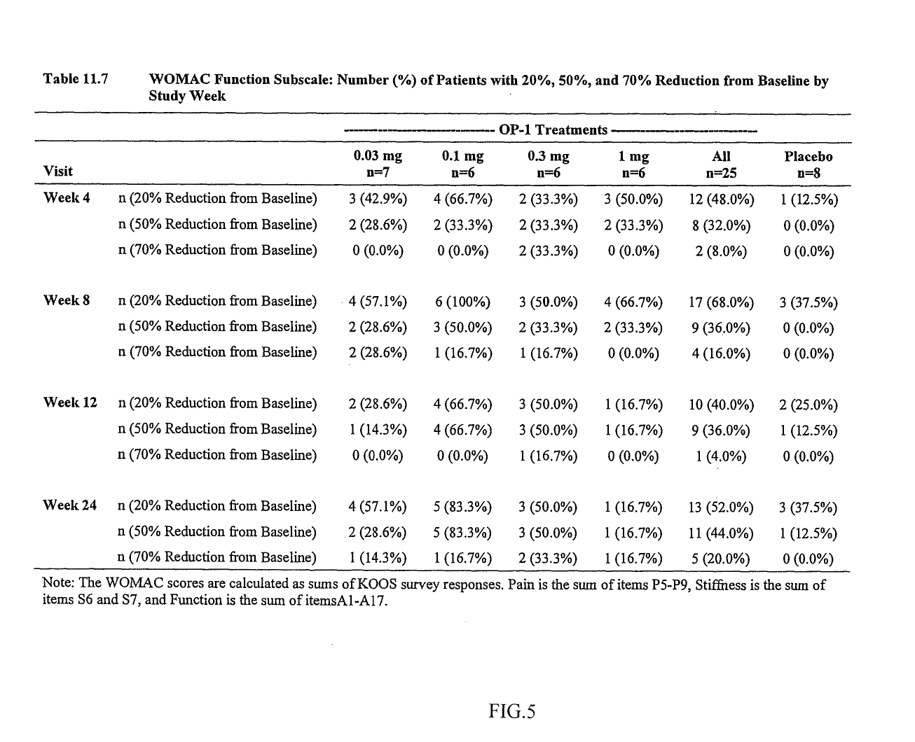

Patients were assessed 4 weeks, 8 weeks, 12 weeks, and 24 weeks from the first administration to determine changes in their WOMAC score. The proportion of subjects with a 20%, 50%, and 70% reduction in the WOMAC function subscale at 4, 8, 12, and 24 weeks is summarized in FIG. 5.

At Weeks 4 and 8, the 0.03, 0.1, 0.3, and 1.0 mg OP-1 groups all had a greater proportion of subjects that achieved a 20% reduction from baseline than the placebo group. At Week 12, the 0.03, 0.1 mg, and 0.3 mg OP-1 groups had a greater proportion of subjects that achieved a 20% reduction than the placebo group. The proportion for the 1.0 mg OP-1 group was lower than the placebo group. At Week 24, the 0.03, 0.1 and 0.3 mg OP-1 groups had a greater proportion of subjects that achieved a 20% reduction than the placebo group. The proportion for the 1.0 mg OP-1 group was lower than the placebo group.

At Weeks 4 and 8, the 0.03, 0.1, 0.3, and 1.0 mg OP-1 groups all had a greater proportion of subjects that achieved a 50% reduction from baseline than the placebo group. At Week 12, the 0.03, 0.1, 0.3, and 1.0 mg OP-1 groups all had a greater proportion of subjects that achieved a 50% reduction than the placebo group. The proportion for the 0.03 mg OP-1 group was lower than the placebo group. At Week 24, the 0.03, 0.1, 0.3, and 1.0 mg OP-1 groups all had a greater proportion of subjects that achieved a 50% reduction than the placebo group.

At Week 4, the 0.3 mg OP-1 group had a greater proportion of subjects that achieved a 70% reduction from baseline than the placebo group. The proportion in the 0.03, 0.1, and 1.0 mg groups was the same as the placebo. At Week 8, the 0.03, 0.1, and 0.3 mg OP-1 groups had a greater proportion of subjects that achieved a 70% reduction than the placebo group. The proportion for the 0.1 mg OP-1 group was the same as the placebo group. At Week 12, the 0.3 mg OP-1 group had a greater proportion of subjects that achieved a 70% reduction than the placebo group. The proportion for the 0.03, 0.1, and 1.0 mg OP-1 groups was the same as the placebo group. At Week 24, the 0.03, 0.1, 0.3, and 1.0 mg groups all had a greater proportion of subjects that achieved at 50% reduction than the placebo group.

In summary, as shown in FIG. 5, while many subjects experienced a 20% improvement in function, the all OP-1 group had a higher proportion of subjects who reached the 50% and 70% reduction endpoints compared to the placebo group. Further, while very few subjects experienced a 70% improvement in function, most subjects in the 0.1 mg and 0.3 mg OP-1 groups maintained a 50% improvement in function at Week 24 which correlated with the groups experiencing the greatest long term pain relief based on the WOMAC pain subscale indicating that these dosages were effective at providing the necessary pain relief to provide concomitant improvements in function.

### Example 8

### Results of OP-1 Administration on the VAS Scale

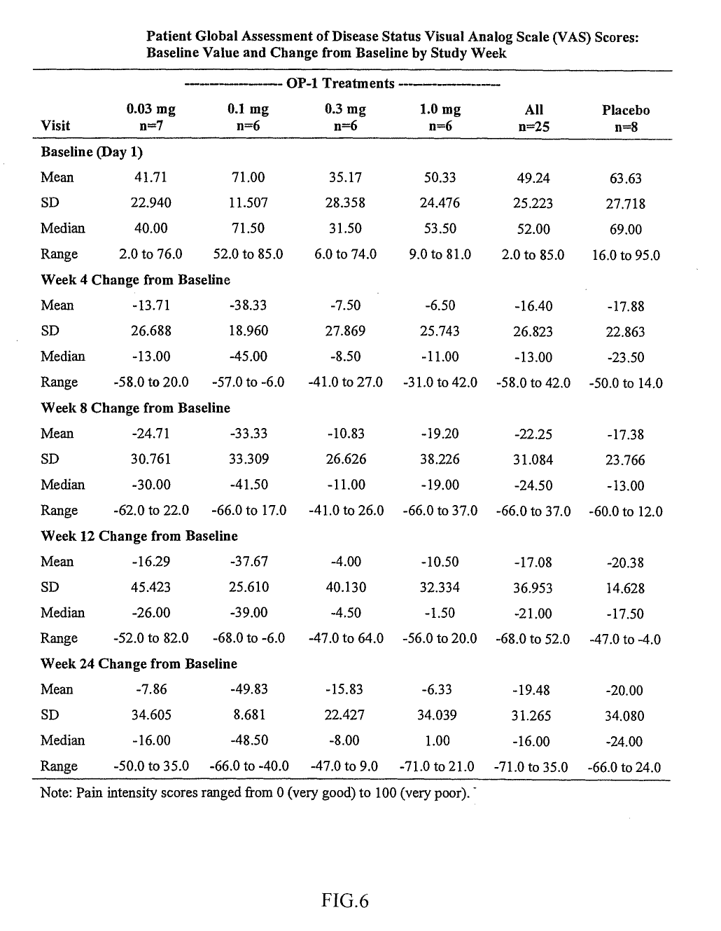

FIG. 6 shows data for the Patient Global Assessment of Disease Status Visual Analog Scale scores. In summary, the patient global assessment scores indicate that all subjects including the placebo group showed some improvement. However, only the 0.1 mg OP-1 group consistently showed more improvement through Week 24 compared to the placebo group.

### Example 9

### Results of OP-1 Administration on the Vas Scale

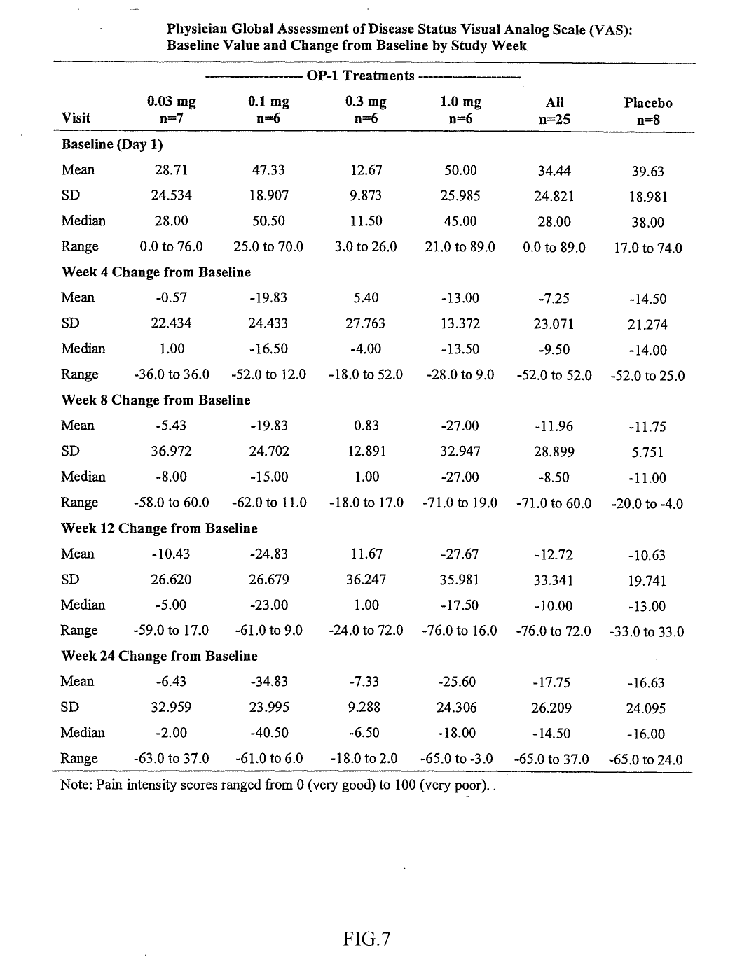

FIG. 7 shows data for the Physician Global Assessment of Disease Status Visual Analog Scale scores. In summary, the patient global assessment scores indicate that the 0.1 mg and 1.0 mg groups showed more improvement than the placebo group through Week 24.

### Example 10

### Pain Reduction in the Hip Joint as a Result of OP-1 Administration

Recombinant OP-1 is prepared as described in Example 2, except that the concentration of OP-1 effective in the knee (0.1 mg) is proportionally adjusted based on the surface area of the hip joint, the volume of the synovial fluid in the hip joint, and the weight of the patient as compared to the knee to arrive at a concentration suitable for injection into the hip. For example, 0.3 mg OP-1 in 1 mL solution is injected intraarticularly into the hip. The patient is assessed for KOOS and WOMAC pain and functionality scores at 4, 8, 12, and 24 weeks and it is determined that the patient experiences a significant reduction in pain and a significant improvement in functionality based on the KOOS and WOMAC scales.

What is claimed:

1. A method for treating osteoarthritis pain in a joint of a subject in need thereof, comprising the step of administering to the joint a dose of a bone morphogenetic protein effective to relieve said osteoarthritis pain.

2. The method of claim 1, wherein the method is further effective to improve functionality in the joint for at least 30 days from the date of administration.

3. The method of claim 1, wherein the dose of bone morphogenetic protein is not effective to induce substantial cartilage growth in the joint.

4. The method of claim 1, wherein the dose of the BMP is administered by an intraarticular injection.

5. The method of claim 1, wherein the dose of the BMP is effective to relieve pain for at least about 30, about 60, about 90, about 120, about 150, about 180 days, or about 360 days.

6. The method of claim 1, further comprising the step of administering a second dose of the BMP to the joint at least 60, 90, 120, 150, 180, 210, 240, 270, 300, 330, or 360 days after the first dose, wherein said second dose of the BMP is effective to relieve osteoarthritis pain for at least 30 days.

7. The method of claim 6, wherein the second dose is equal to the first dose.

8. The method of claim 6, wherein no additional BMP dose is administered to the joint for at least about 60, 90, 120, 150, 180, 210, 240, 270, 300, 330, or 360 days.

9. The method of claim 1, wherein the joint is a knee, hip or shoulder.

10. The method of claim 9, wherein the joint is a knee.

11. The method of claim 1, wherein the BMP is selected from BMP-2, BMP-5, BMP-6, BMP-7, GDF-5, GDF-6 and variants thereof.

12. The method of claim 1, wherein the BMP is BMP-7 or a variant thereof.

13. The method of claim 1, wherein the amount of BMP administered is from about 0.01 mg to about 1.0 mg.

14. The method of claim 1, wherein the amount of BMP administered is from about 0.025 mg to about 0.75 mg.

15. The method of claim 1, wherein the amount of BMP administered is from about 0.05 mg to about 0.5 mg.

16. The method of claim 1, wherein the amount of BMP administered is from about 0.03 mg to about 0.08 mg.

17. The method of claim 1, wherein the amount of BMP administered is from about 0.03 mg to about 0.3 mg.

18. The method of claim 1, wherein the amount of BMP administered is about 0.1 mg.

19. The method of claim 1, wherein the amount of BMP administered is from about 0.08 mg to about 0.4 mg.

20. A method for treating pain associated with osteoarthritis in a joint of a subject in need thereof, comprising the step of intraarticularly administering to the joint a dose of a BMP effective to relieve said pain for at least about 30 days, wherein no additional BMP dose is administered to the joint for at least about 30 days.

21. The method of claim 20, wherein the joint is a knee joint.

22. The method of claim 20, wherein the method is further effective to improve functionality in the joint for at least 30 days.

23. The method of claim 20, wherein the dose of bone morphogenetic protein is not effective to induce substantial cartilage growth in the joint.

24. The method of claim 20, wherein the amount of BMP administered is between about 0.01 mg and 1.0 mg.

25. The method of claim 14, wherein the amount of BMP administered is between about 0.03 mg and 0.3 mg.

26. The method of claim 14, wherein the amount of BMP administered is about 0.1 mg.

27. The method of claim 14, wherein the subject is a human.

28. The method of claim 14, wherein the joint is a knee joint.

29. A method for reducing pain in an osteoarthritic joint of a patient suffering from osteoarthritis comprising administering to the patient a single dose of a BMP wherein a further dose, if administered, is not administered for at least 30 days, wherein said first dose is effective to relieve said osteoarthritis pain for at least 30 days from the time of administration.

30. The method of claim 29, wherein the single dose is injected into the osteoarthritic joint intraarticularly.

31. The method of claim 29, wherein the BMP is OP-1.

32. The method of claim 29, wherein the single dose is about 0.03 mg to about 0.3 mg of BMP.

33. The method of claim 29, wherein the single dose is about 0.1 mg of BMP.

34. The method of claim 29, wherein the joint is a knee joint.
